(19) United States
(12) Patent Application Publication
(10) Pub. No.: US 2007/0010014 A1
(43) Pub. Date: Jan. 11, 2007

(54) Compositions and methods for enhanced delivery to target sites

(75) Inventors: Nichole Lea Wood, Niskayuna, NY (US); Faisal Ahmed Syud, Clifton Park, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: General Electric Company

(21) Appl. No.: 11/175,620

(22) Filed: Jul. 6, 2005

(51) Int. Cl. C12N 15/09

(52) U.S. Cl. 435/455

(57) ABSTRACT

A set of compositions that includes an active agent-labeled species and a pretargeting conjugate is disclosed. The active agent-labeled species includes a first oligomeric nucleotide or mimic thereof conjugated to a first moiety coupled with a diagnostic active agent. The pretargeting conjugate includes a second oligomeric nucleotide or mimic thereof conjugated to a targeting species having a targeting moiety capable of binding to a target or a marker produced by or associated with the target. The second oligomeric nucleotide or mimic thereof has a sequence that is at least partially complementary to a sequence of the first oligomeric nucleotide or mimic thereof. Also disclosed are methods of making the pretargeting conjugate and administering the pretargeting conjugate and active agent-labeled species to a subject for diagnosing a disease condition or assessing the effectiveness of a treatment of the disease condition.

### BACKGROUND OF THE INVENTION

The invention relates to compositions for enhanced delivery to target sites. In particular, the invention relates to such compositions for enhanced delivery of diagnostic agents to disease sites based on a pretargeting strategy.

The growing need for the early diagnosis and assessment and/or treatment of disease can potentially be addressed by compositions that preferentially accumulate at the disease sites. In diagnostic applications, these compositions can elucidate the state of the disease through its distinctive molecular biology expressed as disease markers that are not present, or are present in diminished levels, in healthy tissues. In therapeutic applications, these compositions can deliver an enhanced dose of therapeutic agents to the disease sites through specific interactions with the disease markers. By specifically targeting physiological or cellular functions that are present only in disease states, these compositions can report exclusively on the scope and progress of that disease or exclusively target the diseased tissue. A signal-generating moiety is a key element of these diagnostic compositions, which produce differentiated signals at the disease sites.

The diagnostic detection of a target site benefits from a high signal-to-background ratio of detection agent. Therapy benefits from as high an absolute accretion of therapeutic agent at the target site as possible, as well as a reasonably long duration of binding. In order to improve the targeting ratio and amount of agent delivered to a target site, the use of targeting vectors comprising diagnostic or therapeutic agents conjugated to a targeting moiety for preferential localization has long been known.

Examples of targeting vectors include diagnostic or therapeutic agent conjugates of targeting moieties such as antibody or antibody fragments, cell- or tissue-specific peptides, and hormones and other receptor-binding molecules. For example, antibodies against different determinants associated with pathological and normal cells, as well as associated with pathogenic microorganisms, have been used for the detection and treatment of a wide variety of pathological conditions or lesions. In these methods, the targeting antibody is directly conjugated to an appropriate detecting or therapeutic agent.

One problem encountered in direct targeting methods, i.e., in methods wherein the active agent, such as a diagnostic active agent, is conjugated directly to the targeting moiety and administered simultaneously, is that a relatively small fraction of the conjugate actually binds to the target site, while the majority of conjugate remains in circulation and compromises in one way or another the function of the targeted conjugate (i.e., the conjugate accumulated or bound at the target). In the case of a diagnostic conjugate (e.g., a radioimmunoscintigraphic or magnetic resonance imaging conjugate), the non-targeted conjugate, which remains in circulation, can increase background and decrease resolution.

Pretargeting methods have been developed to increase the target-to-background ratios and improve resolution. In pretargeting methods, a primary targeting species (which is not bound to an active agent) is targeted to an in vivo target site. The primary targeting species comprises a first targeting moiety, which binds to the target site, and a second moiety, which presents a binding site available for binding by a subsequently administered second targeting species. Once sufficient accretion of the primary targeting species is achieved, the second targeting species comprising an active agent and a second targeting moiety, which recognizes the available binding site of the primary targeting species, is administered.

Pretargeting strategy offers certain advantages over the use of direct targeting methods. For example, use of the pretargeting strategy for the in vivo delivery of radionuclides to a target for therapy, e.g., radioimmunotherapy, reduces the marrow toxicity caused by prolonged circulation of a radioimmunoconjugate. This is because the radioisotope is delivered as a rapidly clearing, low molecular weight chelate rather than directly conjugated to a primary targeting molecule, which is often a long-circulating species.

Despite these advantages, known pretargeting strategies still suffer from certain drawbacks. One disadvantage is the very low amount of active agent delivered to the target site compared to when the active agent is directly attached to an antibody, for a variety of reasons. Another disadvantage is that the active agent-carrying vectors, which are often peptides, are often degraded by endogenous proteases in the body. Furthermore, when conjugated to antibodies, the active agent can generate antibodies in a patient.

Consequently, a need still exists for improved diagnostic compositions for use with the pretargeting strategy. It is very desirable to provide such compositions that have fewer side effects to a patient than those exhibited in the prior-art in this area.

### SUMMARY OF THE INVENTION

The purpose and advantages of embodiments of the invention will be set forth and apparent from the description that follows, as well as will be learned by practice of the embodiments of the invention. Additional advantages will be realized and attained by the methods and systems particularly pointed out in the written description and claims hereof, as well as from the appended drawings.

Diagnostic compounds designed for use in a pretargeting strategy comprising an oligomeric nucleotide or mimic thereof that is conjugated to a linker coupled with a diagnostic active agent are disclosed.

Accordingly, one aspect of the invention includes a set of compounds comprising an active agent-labeled species and a pretargeting conjugate. The active agent-labeled species includes a first oligomeric nucleotide or mimic thereof that is conjugated to a linker having a first moiety coupled with a diagnostic active agent. The pretargeting conjugate includes a second oligomeric nucleotide or mimic thereof that is conjugated to a targeting species having a targeting moiety capable of binding to an in-vivo target or a bio-marker produced by or associated with the in-vivo-target. The second oligomeric nucleotide or mimic thereof includes a sequence complementary to at least a portion of the sequence of the first oligomeric nucleotide or mimic thereof; and with the proviso that at least one of the first or second oligomeric nucleotides or mimics thereof is not a morpholino.

A second aspect of the invention includes a method of making a pretargeting conjugate. The method includes reacting a functional group of a crosslinking reagent with a targeting species to form a crosslinking reagent-targeting species complex; and reacting another functional group of the crosslinking reagent with an oligomeric nucleotide or mimic thereof to form a pretargeting conjugate.

A third aspect of the invention includes method of making a pretargeting conjugate. The method includes reacting a functional group of a crosslinking reagent with a targeting species to form a crosslinking reagent-targeting species complex in a buffered aqueous solution at about neutral ph substantially free of primary amines; and reacting another functional group of the crosslinking reagent with an oligomeric nucleotide or mimic thereof to form the pretargeting conjugate.

The accompanying figures, which are incorporated in and constitute part of this specification, are included to illustrate and provide a further understanding of the method and system of the invention. Together with the description, the figures serve to explain the principles of the invention.

### BRIEF DESCRIPTION OF THE DRAWINGS

FIG. 1A is a schematic representation of a pair of active agent-labeled species and pretargeting conjugate in accordance with an embodiment of the invention;

FIG. 1B is another schematic representation of a pair of active agent-labeled species and pretargeting conjugate in accordance with an embodiment of the invention;

FIG. 2 is a schematic representation of a pair of active agent-labeled species and pretargeting conjugate attached to a target in accordance with an embodiment of the invention;

FIG. 3 is a flow chart of a method for diagnosing a disease condition in accordance with an embodiment of the invention;

FIG. 4 is another flow chart of a method for diagnosing a disease condition in accordance with an embodiment of the invention;

FIG. 5 is a schematic representation of a pretargeting conjugate comprising an antibody-peptide nucleic acid (Ab-PNA) in accordance with an embodiment of the invention;

FIG. 6 is a Maldi-time of flight (TOF) MS spectrum of the Ab-PNA pretargeting conjugate shown in FIG. 5 in accordance with an embodiment of the invention;

FIG. 7 is kinetic study demonstrating the binding of the Ab-PNA pretargeting conjugate to a target in accordance with an embodiment of the invention; and

FIG. 8 is a kinetic study demonstrating the coupling of an active agent labeled species and the pretargeting conjugate in accordance with an embodiment of the invention; and

FIG. 9 is a radio-high performance liquid chromatogram (HPLC) of a Cu-64 labeled 1,4,8,11-tetraazacyclotetradecane-N,N′,N″,N′″-tetraacetic acid-cPNA active agent labeled species.

### DETAILED DESCRIPTION OF THE INVENTION

Reference will now be made in detail to exemplary embodiments of the invention, which are illustrated in the accompanying figures and examples. Referring to the drawings in general, it will be understood that the illustrations are for the purpose of describing a particular embodiment of the invention and are not intended to limit the invention thereto.

Whenever a particular embodiment of the invention is said to comprise or consist of at least one element of a group and combinations thereof, it is understood that the embodiment may comprise or consist of any of the elements of the group, either individually or in combination with any of the other elements of that group. Furthermore, when any variable occurs more than one time in any constituent or in formula, its definition on each occurrence is independent of its definition at every other occurrence. Also, combinations of substituents and/or variables are permissible only if such combinations result in stable compounds.

The invention provides diagnostic compounds or pharmaceuticals designed for use in a pretargeting strategy.

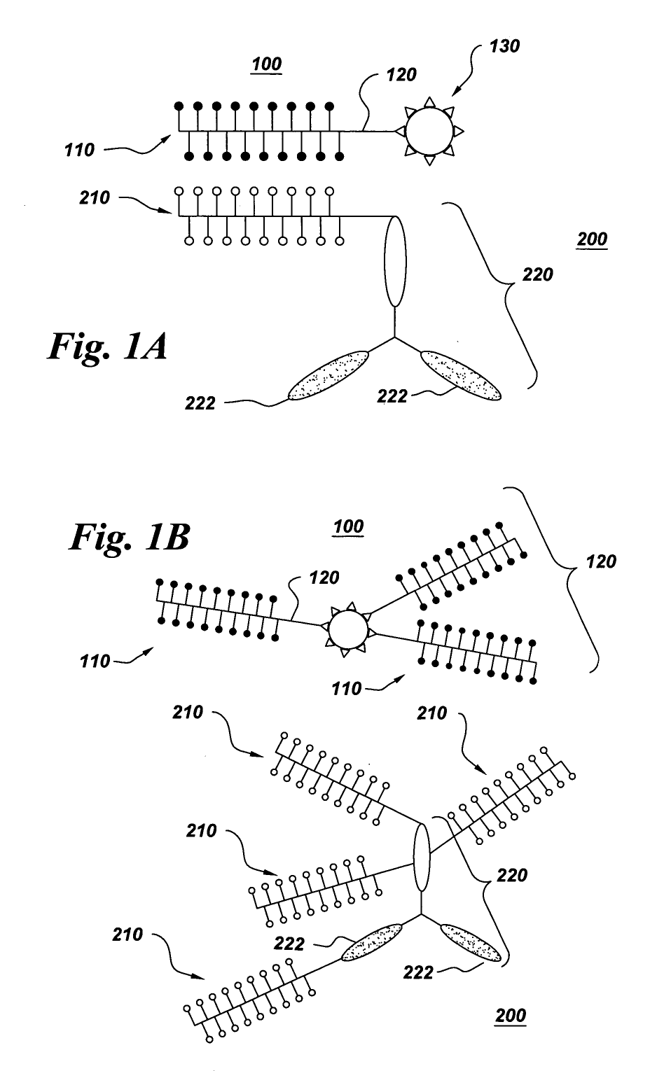

With reference to FIG. 1A, there is shown one embodiment of a set of compounds comprising an active agent-labeled species 100 and a pretargeting conjugate 200. The active agent-labeled species 100 includes a first oligomeric nucleotide or mimic thereof 110 that is conjugated to a linker 120 having a first moiety coupled with a diagnostic active agent 130. The active agent-labeled species 100 may include more than one oligomeric nucleotide or mimics thereof that is conjugated to the linker 120. The pretargeting conjugate 200 includes a second oligomeric nucleotide or mimic thereof 210 that is conjugated to a targeting species 220 having a targeting moiety 222 capable of binding to an in-vivo target or a bio-marker produced by or associated with the in-vivo-target. The second oligomeric nucleotide or mimic thereof 210 includes a sequence complementary to at least a portion of the sequence of the first oligomeric nucleotide or mimic thereof 110 and with the proviso that at least one of the first or second oligomeric nucleotides or mimics thereof is not a morpholino. It should be appreciated that the active agent-labeled species 100 can include one or more oligomeric nucleotides or mimics thereof 110, one or more linkers 120, and one or more diagnostic active agents 130, as shown in FIG. 1B. It should also be appreciated that the pretargeting conjugate 200 can include one or more oligomeric nucleotides or mimic thereof 210, one or more targeting species 220, wherein a targeting species 220 has one or more targeting moieties 222, as shown in FIG. 1B.

Oligomeric Nucleotides or Mimics Thereof

The oligomeric nucleotides or mimics thereof at each occurrence are independent of the oligomeric nucleotides or mimics thereof at every other occurrence, unless otherwise noted. For example, the first and second oligomeric nucleotides or mimics can be the same type or different such as wherein both are oligonucleotides, both are mimics, or one is a mimic and another is an oligonucleotide. Furthermore, when the first and second oligomeric nucleotide or mimics thereof are both oligonucleotides or both mimics thereof, the oligonucleotides or mimics may be of the same or different kind. Furthermore, when an active label agent species or pretargeting conjugate includes more than one oligomeric nucleotide or mimic thereof, the oligomeric nucleotide or mimic thereof at each occurrence is independent of the oligomeric nucleotides or mimics thereof at every other occurrence. Also, combinations of substituents and/or variables are permissible only if such combinations result in stable compounds.

In one embodiment, the first oligomeric nucleotide or mimic thereof is a sequence having the formula of: 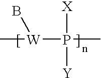
and the second oligomeric nucleotide or mimic thereof is a second oligomeric nucleotide sequence having the formula of: 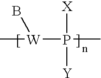
wherein X and Y independently at each occurrence is selected from a group consisting of O, S, OR, BH₃ and NR¹R¹. R independently at each occurrence is selected from a group consisting of a C₁-C₆ alkyl. R¹ and R² independently at each occurrence include a H or C₁-C₆ alkyl, either individually or in combinations thereof. R¹ and R² may optionally form a ring, and the ring may optionally comprise at least one heteroatom. The B (base) independently at each occurrence comprises a natural or synthetic nitrogen-comprising heterocyclic base. Unless otherwise noted, whenever a base is mentioned, the base includes a natural or synthetic base. Examples of nitrogen-comprising heterocyclic bases include, but are not limited to natural nucleoside bases and analogs thereof. Particular example of bases include, but are not limited, uracil, thymine, cytosine, 5-methylcytosine, guanine, 7-deazaguanine, hypoxanthine, 7-deazahypoxanthine, adenine, 7-deazaadenine, 2,6-diaminopurine or analogs or derivatives thereof. Analogs include a substitution or replacement of part of a base. Derivatives include additions to the base. n is an integer in a range from about 4 to about 100. W independently at each occurrence is selected from a group consisting of an acyclic moiety, a carbocyclic moiety, and a natural or a synthetic sugar moiety.

Regarding the acyclic moiety, a carbocyclic moiety, and natural or a synthetic sugar moiety for W, useful carbocyclic moieties have been described by Ferraro, M. and Gotor, V. in Chem Rev. 2000, volume 100, 4319-48. Suitable sugar moieties are described by Joeng, L. S. et al., in J. Med. Chem. 1993, vol. 356, 2627-38; by Kim H. O. et al., in J. Med. Chem. 193, vol. 36, 30-7; and by Eschenmosser A., in Science 1999, vol. 284, 2118-2124. Useful acyclic moieties have been described by Martinez, C. I., et al., in Nucleic Acids Research 1999, vol. 27, 1271-1274 and by Martinez, C. I., et al., in Bioorganic & Medicinal Chemistry Letters 1997, vol. 7, 3013-3016. Non limiting examples of a carbocyclic moiety, a natural or a synthetic sugar moiety, and acyclic moiety for W are shown below

Carbocyclic moiety include the following: 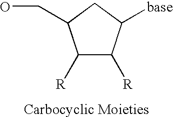
R independently at each occurrence includes H, OH, NHR, F, N₃, SH, SR, or a C₁-C₆ alkyl, aryl, alkyl aryl, and aryl alkyl.

Sugar or synthetic sugars include the following: 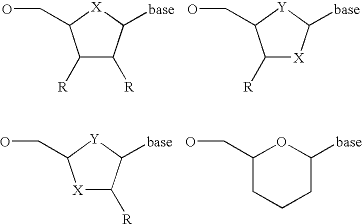
R independently at each occurrence includes H, OH, NHR, F, N₃, SH, SR, or a C₁-C₆ alkyl, aryl, alkyl aryl, and aryl alkyl. X and Y independently at each occurrence include O, S, or NH.

Acyclic moieties include the following: 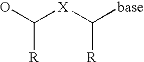
R independently at each occurrence includes H, OH, NHR, F, N₃, SH, SR, or a C₁-C₆ alkyl, aryl, alkyl aryl, and aryl alkyl. X includes O, S, NH, or NR.

In another embodiment, the first oligomeric nucleotide or mimic thereof includes a mimic thereof with a sequence having a formula of: 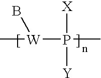
and wherein the second oligomeric nucleotide or mimic thereof also includes a mimic with a sequence having a formula of: 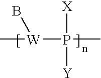
wherein X and Y independently at each occurrence is selected from a group consisting of O, S, OR, BH₃ and NR¹R². R independently at each occurrence is selected from a group consisting of a C₁-C₆ alkyl. R¹ and R² independently at each occurrence include a H or C₁-C₆ alkyl, either individually or in combinations thereof. R¹ and R² may optionally form a ring, and the ring may optionally comprise at least one heteroatom. W independently at each occurrence is selected from a group having an acyclic moiety, a carbocyclic moiety, and a natural or a synthetic sugar moiety. B independently at each occurrence comprises a natural or synthetic nitrogen-comprising heterocyclic base; n is an integer in a range from about 4 to about 100; and with the proviso that X and Y in a terminal unit are not simultaneously O when W is deoxyribose or ribose.

Examples of mimics of the first or second oligomeric nucleotide include, but are not limited to, morpholinos, peptide nucleic acids (“PNAs”), phosphorothioates, 2′-O methyl oligoribonucleotides, oligoboranophosphates, phosphorodithioates, phosphoramidate, phosphorodiamidate, locked nucleic acids, and chimeras, either individually or in combinations thereof. It should be appreciated that the mimics of the first and second oligomeric nucleotides at each occurrence is independent of the mimic at every other occurrence, unless otherwise noted.

In a particular embodiment, the mimic of the first or second oligomeric nucleotides include a peptide nucleic acid (“PNA”) sequence having a formula of: 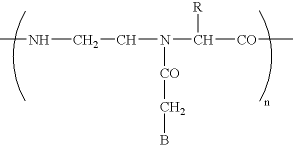
wherein B independently at each occurrence includes a heterocyclic base selected from a group consisting of adenine, guanine, cytosine, thymine, and uracil. It should be appreciated that as previously mentioned, B includes other natural or synthetic bases. R independently at each occurrence is selected from a group consisting of side groups covalently bonded to α-carbons of twenty known α-amino acids and a C₁-C₁₀₀ linear or branched, substituted or unsubstituted alkynyl, alkenyl, alkyl, alkylaryl, aryl, or arylalkyl optionally comprising at least one heteroatom selected from a group consisting of N, O, S, P, and halogens. n is an integer in a range from about 4 to about 30. Particularly, n is an integer in a range from about 10 to about 20. In a particular embodiment, the first and second oligomeric nucleotides or mimic thereof are both PNA mimic sequences, the backbone chain of which comprises repeating units of substituted N-(2-amino-ethyl)-glycine residues, as described above. The second oligomeric nucleotide or mimic thereof comprises a second PNA sequence that is complementary to the first PNA sequence. The second PNA sequence binds to the first PNA sequence by base pairing; i.e., A forms hydrogen bonding with T (or U), and G with C.

In another embodiment of the mimic, the mimic of the first or second oligomeric nucleotides include a morpholino sequence having a formula of: 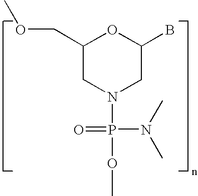
wherein B independently at each occurrence is a heterocyclic base selected from a group consisting of adenine, guanine, cytosine, thymine, and uracil; and n is an integer in a range from about 4 to about 100.

In one embodiment, the mimic of the first or second oligomeric nucleotide includes a phosphorothioate sequence having a formula of: 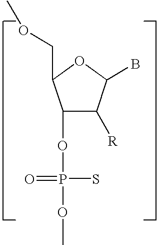
wherein B independently at each occurrence is a heterocyclic base selected from a group consisting of adenine, thymine, uracil, guanine, cytosine, and 5-methyl cytosine; n is an integer in a range from about 6 to about 100; and wherein R independently at each occurrence is selected from a group consisting of H, OH and OCH₃. In a particular embodiment, n in a range from about 15 to about 35 and R is H.

In another embodiment, the mimic of the first or second oligomeric nucleotides comprises a 2′-O methyl oligoribonucleotide sequence having a formula of: 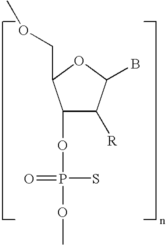
wherein B independently at each occurrence is a heterocyclic base selected from a group consisting of adenine, thymine, uracil, guanine, cytosine, and 5-methyl cytosine; n is an integer in a range from about 6 to about 100 and particularly in a range from about 15 to about 35; and R independently at each occurrence comprises OR′, wherein R′ comprises C₁-C₆ alkyl chain with 0 to about 2 heteroatoms. Particularly, R¹ comprises CH₃.

In yet another embodiment, the mimic of the first or second oligomeric nucleotides comprises a chimeric oligonucleotide sequence having a formula of: 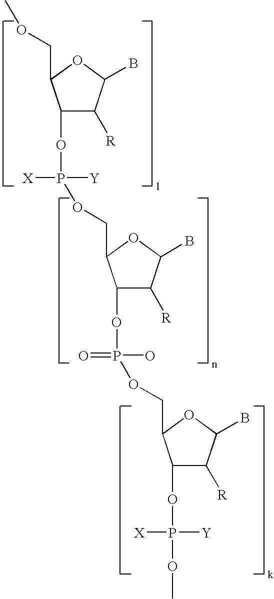
wherein B independently at each occurrence is a heterocyclic base selected from a group consisting of adenine, thymine, uracil, guanine, cytosine, and 5-methyl cytosine; X and Y are independently at each occurrence selected from a group consisting of O, S, OR, and BH₃; R independently at each occurrence is H, OH, or OCH₃; 1 is an integer in a range from about 2 to about 20; k is an integer in a range from about 2 to about 20; and n is an integer in a range from about 1 to about 20.
Active Agent Label Species

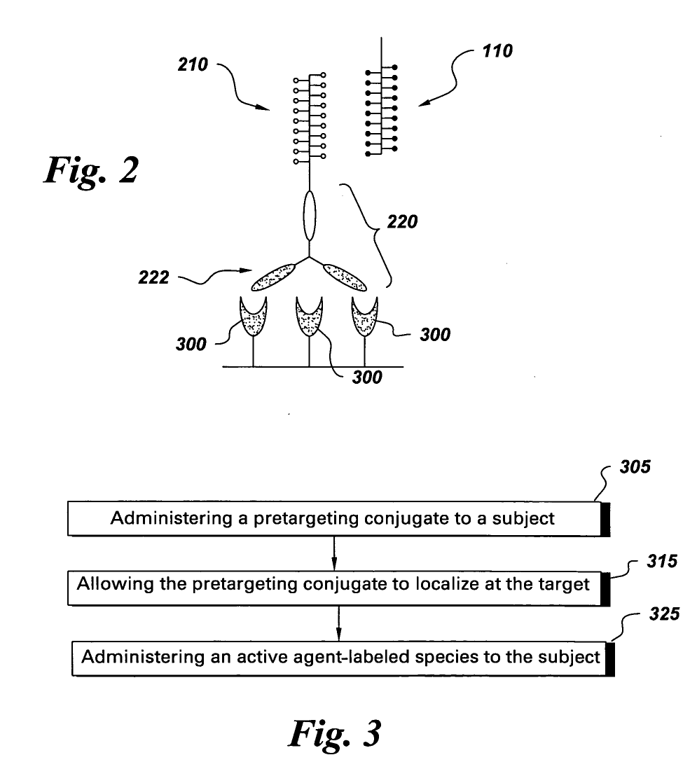

Embodiments of the active agent labeled species are shown in FIG. 1 and FIG. 2 and represented by the schema below:

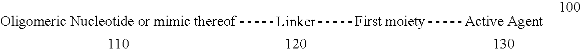

The linker includes any linking moiety that attaches the oligonucleotide or mimic thereof to the diagnostic active agent through a first moiety. The linker may attach to the oligonucleotide or mimic thereof at any location, such as but not limited to, the N terminus, C terminus or any other location. The linker can be as short as one carbon or a long polymeric species such as polyethylene glycol, polylysine or other polymeric species normally used in the pharmaceutical industry for modulating pharmacokinetic and biodistribution characteristics of active agents. Other linkers of varying length include C₁-C₂₅₀ length with one or more heteroatoms selected from O, S, N, P, and optionally substituted with halogen atoms.

The first moiety may simply be an extension of the linker, formed by the reaction of a reactive species on the linker with a reactive group on the active agent, or a chelator that complexes the active agent. Examples of reactive species and the reactive group include, but are not limited to, activated esters (such as N-hydroxysuccinimide ester, pentafluorophenyl ester), a phosphoramidite, an isocyanate, an isothiocyanate, an aldehyde, an acid chloride, a sulfonyl chloride, a maleimide an alkyl halide, an amine, a phosphine, a phosphate, an alcohol or a thiol with the proviso that the reactive species and reactive group are matched to undergo a reaction yielding covalently linked conjugates.

In a particular embodiment, the linker comprises at least one of an oligomeric or polymeric species made of natural or synthetic monomers, oligomeric or polymeric moiety selected from a pharmacologically acceptable oligomer or polymer composition, an oligo- or poly-amino acid, peptide, saccharide, a nucleotide, and an organic moiety with 1-250 carbon atoms, either individually or in combination thereof. The organic moiety with 1-250 carbon may contain one or more heteroatoms such as O, S, N or P and optionally substituted with halogen atoms at one or more places.

The first moiety comprises a chelating moiety that is conjugated to the oligomeric or polymeric linker species, and the diagnostic active agent is capable of generating a signal that is detectable.

Diagnostic Active Agents

Diagnostic active agents include a procedure, composition of matter or device (or combination of them) that conveys information about a disease, condition or disorder affecting an organism. Examples of diagnostic active agents include radioisotopes. Suitable radioisotopes for coupling with the first oligomeric nucleotide or mimic thereof to produce diagnostic active agents and use in diagnostic methods include, but are not limited to, actinium-225, astatine-211, iodine-120, iodine-123, iodine-124, iodine-125, iodine-126, iodine-131, iodine-133, bismuth-212, arsenic-72, bromine-75, bromine-76, bromine-77, indium-110, indium-111, indium-113m, gallium-67, gallium-68, strontium-83, zirconium-89, ruthenium-95, ruthenium-97, ruthenium-103, ruthenium-105, mercury-107, mercury-203, rhenium-186, rhenium-188, tellurium-121m, tellurium-122m, tellurium-125m, thulium-165, thulium-167, thulium-168, technetium-94m, technetium-99m, fluorine-18, silver-111, platinum-197, palladium-109, copper-62, copper-64, copper-67, phosphorus-32, phosphorus-33, yttrium-86, yttrium-90, scandium-47, samarium-153, lutetium-177, rhodium-105, praseodymium-142, praseodymium-143, terbium-161, holmium-166, gold-199, cobalt-57, cobalt-58, chromium-51, iron-59, selenium-75, thallium-201, and ytterbium-169. Particularly, the radioisotope will emit a particle or ray in the 10-7,000 keV range, more particularly 50-1,500 keV.

Particular examples of diagnostic active agent include, but are not limited to, the following isotopes: F-18, I-123, I-124, I-125, Cu-64, Cu-67, At-211, I-120, I-123, I-124, I-125, As-72, Br-75, Br-76, Br-77, In-110, In-111, In-113m, G Ga-68, Lu-177, Ti-201, Yb-169, Rb-82, Co-55, Cu-61, As-70, As-71, As-74, Y-88, C-11, N-13, Cu-60, O-15, Zr-89 and Ga-66.

Isotopes for imaging applications include: iodine-123, iodine-125, iodine-131, indium-111, gallium-67, ruthenium-97, technetium-99m, cobalt-57, cobalt-58, chromium-51, iron-59, selenium-75, thallium-201, ytterbium-169, copper -64, and fluorine-18.

In one aspect, the diagnostic active agent is a magnetic resonance imaging contrast agent, which enhances the contrast of images obtained in magnetic resonance imaging procedure. Suitable paramagnetic ions that are useful for magnetic resonance imaging (“MRI”) are those of elements having atomic numbers of 21-29, 42, 44, and 58-70. Particularly useful are gadolinium ion and iron metal, ion, or oxides. Particularly, gadolinium ions are bound by chelators, such as polycarboxylic acids (carboxylic acids having a plurality of —COOH groups), which are conjugated directly or indirectly to the first oligomeric nucleotide or mimic thereof through one of the —CO(O)— groups. Non-limiting examples of such chelators are diethylenetriamine-pentaacetic acid (“DTPA”); 1,4,7,10-tetraazacyclododecane-N,N′,N″,N′″-tetraacetic acid (“DOTA”); p-isothiocyanatobenzyl-1,4,7,10-tetraazacyclododecane-1,4,7,10-tetraacetic acid (“p-SCN-Bz-DOTA”); 1,4,7,10-tetraazacyclododecane-N,N′,N″-triacetic acid (“DO3A”); 1,4,7,10-tetraazacyclododecane-1,4,7,10-tetrakis(2-propionic acid) (“DOTMA”); 3,6,9-triaza -12-oxa-3,6,9-tricarboxymethylene-10-carboxy-13-phenyl-tridecanoic acid (“B -19036”); 1,4,7-triazacyclononane-N,N′,N″-triacetic acid (“NOTA”); 1,4,8,11-tetraazacyclotetradecane-N,N′,N″,N′″-tetraacetic acid (“TETA”); triethylene tetraamine hexaacetic acid (“TTHA”); trans-1,2-diaminohexane tetraacetic acid (“CYDTA”); 1,4,7,10-tetraazacyclododecane-1-(2-hydroxypropyl)4,7,10-triacetic acid (“HP-DO3A”); trans-cyclohexane-diamine tetraacetic acid (“CDTA”); trans(1,2)-cyclohexane diethylene triamine pentaacetic acid (“CDTPA”); 1-oxa-4,7,10-triazacyclododecane-N,N′,N″-triacetic acid (“OTTA”); 1,4,7,10-tetraazacyclododecane-1,4,7,10-tetrakis {3-(4-carboxyl)-butanoic acid}; 1,4,7,10-tetraazacyclododecane-1,4,7,10-tetrakis(acetic acid-methyl amide); 1,4,7,10-tetraazacyclododecane-1,4,7,10-tetrakis(methylene phosphonic acid); and derivatives thereof.

Superparamagnetic metal oxides, such as iron, chromium, cobalt, manganese, nickel, and tungsten oxide, are also suitable for generating magnetic resonance signal useful for diagnostic purposes, as disclosed, for example, in U.S. Pat. Nos. 5,492,814 and 5,314,679. Such metal oxides are particularly present in nanometer-sized aggregates (e.g. from about 10 nm to about 500 nm), either uncoated or particularly coated with a shell comprising a biologically compatible material, such as polysaccharide, poly(amino acid), or organosilane. The coated or uncoated metal oxide aggregates are then covalently attached directly or indirectly (through a linker) to the first oligomeric nucleotide or mimic thereof to produce an MRI signal generating species. Particularly, the first oligomeric nucleotide or mimic thereof is a PNA, and the attachment is effected at the terminal amino or carboxylic group of the PNA sequence. Methods for attachment of metal oxide (such as iron oxide) particles to organic materials, such as proteins, are known and disclosed, for example, in U.S. Pat. Nos. 5,492,814 and 4,628,037.

An MRI Active Agent-Labeled Species

In a particular embodiment, the linker includes an oligomeric or polymeric species comprising polylysine having from about 100 to about 600 lysine residues, and wherein at least ninety percent of the lysine residues are conjugated to a chelating moiety. A plurality of amino acid residues of the poly(amino acid) is conjugated to chelators that form coordination complexes with paramagnetic ions. Suitable chelators include but are not limited to, diethylenetriamine-pentaacetic acid (“DTPA”), 1,4,7,10-tetraazacyclododecane-N,N′,N″,N′″-tetraacetic acid (“DOTA”), p-isothiocyanatobenzyl-1,4,7,10-tetraazacyclododecane-1,4,7,10-tetraacetic acid (“p-SCN-Bz-DOTA”), 1,4,7,10-tetraazacyclododecane-N,N′,N″-triacetic acid (“DO3A”), 1,4,7,10-tetraazacyclododecane-1,4,7,10-tetrakis(2-propionic acid) (“DOTMA”), 3,6,9-triaza-12-oxa-3,6,9-tricarboxymethylene-10-carboxy-13-phenyl-tridecanoic acid (“B-19036”), 1,4,7-triazacyclononane-N,N′,N″-triacetic acid (“NOTA”), 1,4,8,11-tetraazacyclotetradecane-N,N′,N″,N′″-tetraacetic acid (“TETA”), triethylene tetraamine hexaacetic acid (“TTHA”), trans-1,2-diaminohexane tetraacetic acid (“CYDTA”), 1,4,7,10-tetraazacyclododecane-1-(2-hydroxypropyl)4,7,10-triacetic acid (“HP-DO3A”), trans-cyclohexane-diamine tetraacetic acid (“CDTA”), trans(1,2)-cyclohexane diethylene triamine pentaacetic acid (“CDTPA”), 1-oxa-4,7,10-triazacyclododecane-N,N′,N″-triacetic acid (“OTTA”), 1,4,7,10-tetraazacyclododecane-1,4,7,10-tetrakis {3-(4-carboxyl)-butanoic acid}, 1,4,7,10-tetraazacyclododecane-1,4,7,10-tetrakis(acetic acid-methyl amide), 1,4,7,10-tetraazacyclododecane-1,4,7,10-tetrakis(methylene phosphonic acid), and derivatives thereof, either individually or in combinations thereof.

As yet another alternative, the active agent-labeled species comprises a poly(amino acid) chain (e.g., polylysine), wherein at least ninety percent of the free amino side groups are conjugated to a carboxylic acid having a plurality of carboxylic groups, and wherein one or more of the remaining free amino side groups are conjugated to a PET or SPECT signal-generating moiety; e.g., 4-iodobenzamide, 4-fluorobenzamide, or a chelating moiety that forms a coordination complex with a PET or SPECT signal-generating metal ion. The chelating moiety can be TETA or one of the carboxylic acids having a plurality of carboxylic groups, as disclosed above.

Pretargeting Conjugate

The targeting species 220 that is conjugated to the second oligomeric nucleotide or mimic thereof to form the pretargeting conjugate can be a compound or a fragment of a compound. As shown in FIG. 2, the targeting species 220 has one or more targeting moieties 222 that binds to a target site 300 or to a substance produced by or associated with the target site via a primary binding site. Furthermore, as shown in FIG. 2, the targeting species may bind to one or more in-vivo targets 300 or a biomarker produced by or associated with the in-vivo-target. The target site is a specific site to which the diagnostic agent is to be delivered, such as a cell or group of cells, tissue, organ, tumor, or lesion. The targeting moiety binds to the target site or to a substance produced by or associated with the target site via a primary binding site. Non-limiting examples of targeting moieties include, but are not limited to, proteins, peptides, polypeptides, glycoproteins, lipoproteins, phospholipids, oligonucleotides, steroids, alkaloids or the like, e.g., hormones, lymphokines, growth factors, albumin, cytokines, enzymes, immune modulators, receptor proteins, oligonucleotides or mimics thereof, and antibodies and antibody fragments, either individually or in any combination thereof as well as derivatives thereof. Examples of particular targeting species include aptamers and thioaptemers. The targeting moieties preferentially bind marker substances that are produced by or associated with the target site.

The targeting species also has a separate functional group that is capable of forming a bond with the second oligomeric nucleotide or mimic thereof. In one embodiment, such a bond may be an amide bond formed by the reaction of a carboxylic acid group in the targeting species and the terminal amino group in the second oligomeric nucleotide or mimic thereof (or the reaction of an amino group in the targeting species and the terminal carboxylic acid group in the second oligomeric nucleotide or mimic thereof). In a particular embodiment, the second oligomeric nucleotide or mimic thereof is a PNA that is at least partially complementary to a PNA in the active agent-labeled species.

Proteins are known that preferentially bind marker substances that are produced by or associated with lesions. For example, antibodies can be used against cancer-associated substances, as well as against any pathological lesion that shows an increased or unique antigenic marker, such as against substances associated with cardiovascular lesions, for example, vascular clots including thrombi and emboli, myocardial infarctions and other organ infarcts, and atherosclerotic plaques; inflammatory lesions; and infectious and parasitic agents.

Cancer states include carcinomas, melanomas, sarcomas, neuroblastomas, leukemias, lymphomas, gliomas, myelomas, and neural tumors.

Infectious diseases include those caused by invading microbes or parasites. As used herein, “microbe” denotes virus, bacteria, rickettsia, mycoplasma, protozoa, fungi and like microorganisms, “parasite” denotes infectious, generally microscopic or very small multicellular invertebrates, or ova or juvenile forms thereof, which are susceptible to antibody-induced clearance or lytic or phagocytic destruction, e.g., malarial parasites, spirochetes and the like, including helminths, while “infectious agent” or “pathogen” denotes both microbes and parasites.

The protein substances useful as targeting species include, but are not limited to, protein, peptide, polypeptide, glycoprotein, lipoprotein, hormones, lymphokines, growth factors, albumin, cytokines, enzymes, immune modulators, receptor proteins, antibodies and antibody fragments, soluble and serum proteins, proteins expressed on a surface of a cell, segment of proteins that are or can be made water-soluble, non-immunoglobulin proteins, intracellular proteins, and derivatives thereof.

The protein substances of particular interest in the invention are antibodies and antibody fragments. The terms “antibodies” and “antibody fragments” mean generally immunoglobulins or fragments thereof that specifically bind to antigens to form immune complexes.

The antibody may be a whole immunoglobulin of any class; e.g., IgG, IgM, IgA, IgD, IgE, chimeric or hybrid antibodies with dual or multiple antigen or epitope specificities. It can be a polyclonal antibody, particularly a humanized or an affinity-purified antibody from a human. It can be an antibody from an appropriate animal; e.g., a primate, goat, rabbit, mouse, or the like. If the target site-binding region is obtained from a non-human species, it is preferred that the target species is humanized to reduce immunogenicity of the non-human antibodies, for use in human diagnostic or therapeutic applications. Such a humanized antibody or fragment thereof is also termed “chimeric.” For example, a chimeric antibody comprises non-human (such as murine) variable regions and human constant regions. A chimeric antibody fragment can comprise a variable binding sequence or complementarity-determining regions (“CDR”) derived from a non-human antibody within a human variable region framework domain. Monoclonal antibodies are also suitable because of their high specificities. Monoclonal antibodies are readily prepared by what are now considered conventional procedures of immunization of mammals with an immunogenic antigen preparation, fusion of immune lymph or spleen cells with an immortal myeloma cell line, and isolation of specific hybridoma clones. More unconventional methods of preparing monoclonal antibodies are also included, such as interspecies fusions and genetic engineering manipulations of hypervariable regions, since it is primarily the antigen specificity of the antibodies that affects their utility. It will be appreciated that newer techniques for production of monoclonal antibodies (“MAb”) can also be used; e.g., human MAbs, interspecies MAbs, chimeric (e.g., human/mouse) MAbs, genetically engineered antibodies, and the like.

Useful antibody fragments include F(ab′)₂, F(ab)₂, Fab′, Fab, Fv, and the like including hybrid fragments. Particular fragments are Fab′, F(ab′)₂, Fab, and F(ab)₂. Also useful are any subfragments retaining the hypervariable, antigen-binding region of an immunoglobulin and having a size similar to or smaller than a Fab′ fragment. An antibody fragment can include genetically engineered and/or recombinant proteins, whether single-chain or multiple-chain, which incorporate an antigen-binding site and otherwise function in vivo as targeting species in substantially the same way as natural immunoglobulin fragments. Such single-chain binding molecules are disclosed in U.S. Pat. No. 4,946,778. Fab′ antibody fragments may be conveniently made by reductive cleavage of F(ab′)₂ fragments, which themselves may be made by pepsin digestion of intact immunoglobulin. Fab antibody fragments may be made by papain digestion of intact immunoglobulin, under reducing conditions, or by cleavage of F(ab)₂ fragments which result from careful papain digestion of whole immunoglobulin. The fragments may also be produced by genetic engineering.

It should be noted that mixtures of antibodies and immunoglobulin classes can be used, as can hybrid antibodies. Multispecific, including bispecific and hybrid, antibodies and antibody fragments are sometimes desirable for detecting and treating lesions and comprise at least two different substantially monospecific antibodies or antibody fragments, wherein at least two of the antibodies or antibody fragments specifically bind to at least two different antigens produced or associated with the targeted lesion or at least two different epitopes or molecules of a marker substance produced or associated with the targeted lesion. Multispecific antibodies and antibody fragments with dual specificities can be prepared analogously to the anti-tumor marker hybrids disclosed in U.S. Pat. No. 4,361,544. Other techniques for preparing hybrid antibodies are disclosed in; e.g., U.S. Pat. Nos. 4,474,893 and 4,479,895, and in Milstein et al., Immunology Today, Vol. 5, 299 (1984).

Particular proteins that may be used are proteins having a specific immunoreactivity to a biomarker substance of at least 60% and a cross-reactivity to other antigens or non-targeted substances of less than 35%.

As disclosed above, antibodies against tumor antigens and against pathogens are known. For example, antibodies and antibody fragments which specifically bind biomarkers produced by or associated with tumors or infectious lesions, including viral, bacterial, fungal and parasitic infections, and antigens and products associated with such microorganisms have been disclosed in Hansen et al. (U.S. Pat. No. 3,927,193) and Goldenberg (U.S. Pat. Nos. 4,331,647, 4,348,376, 4,361,544, 4,468,457, 4,444,744, 4,818,709 and 4,624,846). In particular, antibodies against an antigen, e.g., a gastrointestinal, lung, breast, prostate, ovarian, testicular, brain or lymphatic tumor, a sarcoma, or a melanoma, are advantageously used.

A wide variety of monoclonal antibodies against infectious disease agents have been developed, and are summarized in a review by Polin, in Eur. J. Clin. Microbiol., 3(5):387-398, 1984, showing ready availability. These include MAbs against pathogens and their antigens. Exemplary infectious disease agents are disclosed in U.S. Pat. No. 5,482,698.

Additional examples of MAbs generated against infectious organisms that have been described in the literature are noted below.

MAbs against the gp 120 glycoprotein antigen of human immunodeficiency virus 1 (HIV-1) are known, and certain of such antibodies can have an immunoprotective role in humans. See, e.g., Rossi et al., Proc. Natl. Acad. Sci. USA, Vol. 86, pp. 8055-58 (1990). Other MAbs against viral antigens and viral-induced antigens are also known. MAbs against malaria parasites can be directed against the sporozoite, merozoite, schizont and gametocyte stages.

Suitable MAbs have been developed against most of the microorganisms (bacteria, viruses, protozoa, other parasites) responsible for the majority of infections in humans, and many have been used previously for in vitro diagnostic purposes. These antibodies, and newer MAbs that can be generated by conventional methods, are also appropriate for use.

Proteins useful for detecting and/or treating cardiovascular lesions include fibrin-specific proteins; for example, fibrinogen, soluble fibrin, antifibrin antibodies and fragments, fragment E₁ (a 60 kDa fragment of human fibrin made by controlled plasmin digestion of crosslinked fibrin), plasmin (an enzyme in the blood responsible for the dissolution of fresh thrombi), plasminogen activators (e.g., urokinase, streptokinase and tissue plasminogen activator), heparin, and fibronectin (an adhesive plasma glycoprotein of 450 kDa) and platelet-directed proteins; for example, platelets, antiplatelet antibodies, and antibody fragments, anti-activated platelet antibodies, and anti-activated platelet factors, which have been reviewed by Koblik et al., Semin. Nucl. Med., Vol. 19, 221-237 (1989).

In one embodiment, the targeting species is an MAb or a fragment thereof that recognizes and binds to a heptapeptide of the amino terminus of the β-chain of fibrin monomer. Fibrin monomers are produced when thrombin cleaves two pairs of small peptides from fibrinogen. Fibrin monomers spontaneously aggregate into an insoluble gel, which is further stabilized to produce blood clots. The second oligomeric nucleotide or mimic thereof of the pretargeting conjugate of the invention is attached to the MAb or fragment thereof containing the binding site for this heptapeptide can provide an effective agent for the detection, localization, or therapy of deep vein and coronary artery thrombi. Such heptapeptide and MAb are disclosed in K. Y. Hui et al., “Monoclonal Antibodies to a Synthetic Fibrin-Like Peptide Bind to Human Fibrin but Not Fibrinogen,” Science, Vol. 222, 1129 (1983). In a particular embodiment, the second oligomeric nucleotide or mimic thereof is at least partially complementary to a PNA in the active agent-labeled species.

In another embodiment, the targeting species is a chimeric antibody derived from an antibody designated as NR-LU-10. This chimeric antibody has been designated as NR-LU-13 and disclosed in U.S. Pat. No. 6,358,710. NR-LU-13 contains the murine Fv region of NR-LU-10 and therefore comprises the same binding specificity as NR-LU-10. It also comprises human constant regions. Thus, this chimeric antibody binds the NR-LU-10 antigen and is less immunogenic because it is made more human-like. NR-LU-10 is a nominal 150 kilodalton (or kDa) murine IgG_{2b} pan carcinoma monoclonal antibody that recognizes an approximately 40 kDa glycoprotein antigen expressed on most carcinomas, such as small cell lung, non-small cell lung, colon, breast, renal, ovarian, pancreatic, and other carcinoma tissues. The NR-LU-10 antigen has been further described by Varki et al., “Antigens Associated With a Human Lung Adenocarcinoma Defined by Monoclonal Antibodies,” Cancer Research, Vol. 44, 681-87 (1984), and Okabe et al., “Monoclonal Antibodies to Surface Antigens of Small Cell carcinoma of the Lungs,” Cancer Research Vol. 44, 5273-78 (1984). Methods for preparing antibodies that binds to epitopes of the NR-LU-10 antigen are known and are disclosed in U.S. Pat. No. 5,084,396. One suitable method for producing monoclonal antibodies is the standard hybridoma production and screening process, which is well known in the art. In a particular embodiment, the targeting species is a humanized antibody or humanized antibody fragment that binds specifically to the antigen bound by antibody NR-LU-13. A humanization method comprises grafting only non-human CDRs onto human framework and constant regions (see; e.g., Jones et al., Nature, Volume 321, 522-35 (1986)). Another humanization method comprises transplanting the entire non-human variable domains, but cloaking (or veneering) these domains by replacement of exposed residues reduce immunogenicity (see; e.g., Padlan, Molec. Immun., Vol. 28, 489-98 (1991)). Exemplary humanized light and heavy sequences derived from the light and heavy sequences of the NR-LU-13 antibody are disclosed in U.S. Pat. No. 6,358,710, and are denoted therein as NRX451. The phrase “binds specifically” with respect to antibody or antibody fragment means such antibody or antibody fragment has a binding affinity of at least about 10⁴ M⁻¹. Particularly, the binding affinity is at least about 10⁶ M⁻¹, and more particularly, at least about 10⁸ M⁻¹.

According to still another embodiment, the targeting species is a humanized anti-p185^{HER2} antibody that specifically recognizes the p185 HER2 protein expressed on breast cancer cells. A humanized anti-p185^{HER2} antibody known as Herceptin is widely available. An anti-HER2 murine MAb known as ID5 is available from Applied BioTechnology/Oncogene Science (Cambridge, Mass.), which can be humanized according to conventional methods. See, e.g., X. F. Lee et al., “Differential Signaling by an Anti-p185^{HER2} Antibody and Hergulin,” Cancer Research, Vol. 60, 3522-31 (2000).

In other embodiments, the targeting species is an antibody or a fragment thereof, particularly a human or humanized antibody or fragment thereof, that is raised against one of anti-carcinogembryonic antigen (“CEA”), anti-colon-specific antigen-p (“CSAp”), and other well known tumor-associated antigens, such as CD19, CD 20, CD21, CD22, CD23, CD30, CD74, CD80, HLA-DR, I, MUC 1, MUC 2, MUC 3, MUC 4, EGFR, HER2/neu, PAM-4, Bre3, TAG-72 (C72.3, CC49), EGP-1 (e.g., RS7), EGP-2 (e.g., 17-1A and other Ep-CAM targets), Le(y(e.g., B3), A3, KS-1, S100, IL-2, T101, necrosis antigens, folate receptors, angiogenesis markers (e.g., VEGFR), tenascin, PSMA, PSA, tumor-associated cytokines, MAGE and/or fragments thereof. Tissue-specific antibodies (e.g., against bone marrow cells, such as CD34, CD74, etc., parathyroglobulin antibodies, etc.) as well as antibodies against non-malignant diseased biomarkers, such as macrophage antigens of atherosclerotic plaques (e.g., CD74 antibodies), and also specific pathogen antibodies (e.g., against bacteria, viruses, and parasites) are well known in the art.

It should be understood that the foregoing disclosure of various antigens or biomarkers that can be used to raise specific antibodies against them (and from which antibodies fragments may be prepared) serves only as examples, and is not to be construed in any way as a limitation of the invention.

Conjugating or Attaching Targeting Species to an Oligomeric Nucleotide or Mimic Thereof

Another aspect of the invention provides a method of making a pretargeting conjugate. The method includes reacting a functional group of a crosslinking reagent with a targeting species to form a crosslinking reagent-targeting species complex; and reacting another functional group of the crosslinking reagent with an oligomeric nucleotide or mimic thereof to form a pretargeting conjugate.

In a particular embodiment, the oligomeric nucleotide or mimic thereof comprises a sequence having a formula of: 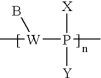
wherein X and Y independently at each occurrence is selected from a group consisting of O, S, OR, BH, and NR¹R¹. R independently at each occurrence is selected from a group consisting of a C₁-C₆ alkyl. R¹ and R² independently at each occurrence include a H or C₁-C₆ alkyl, either individually or in combinations thereof. R¹ and R² may optionally form a ring, and the ring may optionally comprise at least one heteroatom. W independently at each occurrence is selected from a group consisting of an acyclic moiety, a carbocyclic moiety, and a natural or a synthetic sugar moiety; wherein B independently at each occurrence comprises a synthetic or natural nitrogen-comprising heterocyclic base; and wherein n is an integer in a range from about 4 to about 100.

In one embodiment, reacting the functional group of the crosslinking reagent with the targeting species comprises incubating the functional group of the crosslinking reagent with the targeting species. Reacting the functional group of the crosslinking reagent with the targeting species also further comprises the formation of a covalent bond between the crosslinking reagent and the targeting species. In a particular embodiment, the functional group of the cross-linking reagent is reacted with the targeting species in a buffered aqueous solution maintained at a pH from about 6.5 to about 7.5. In a more particular embodiment, the pH is about neutral and the buffered aqueous solution is substantially free of primary amines. Substantially free of primary amines means the primary amine concentration in the buffered aqueous solution is less than about 5 mM.

In another embodiment, the cross-linking reagent is provided in excess of the targeting species. The invention further comprises removing any excess unreacted crosslinking reagent. The excess unreacted crosslinking reagent may be removed by any conventional means known to one of ordinary skill in the art. The method further comprises inhibiting the reaction between the functional group of the crosslinking reagent and the targeting species by providing primary amine.

Regarding reacting the another functional group of the crosslinking reagent with the oligomeric nucleotide or mimic thereof, in one embodiment, the crosslinking reagent is incubated with the oligomeric nucleotide or mimic thereof. Reacting the another functional group of the crosslinking reagent with the oligomeric nucleotide or mimic thereof also includes the formation of a covalent bond between the crosslinking reagent and the oligomeric nucleotide or mimic thereof. In a particular embodiment, the reaction of the another functional group with the oligomeric nucleotide or mimic thereof occurs in a buffered aqueous solution maintained at a pH from about 6.5 to about 7.5. In a more particular embodiment, the pH is about neutral and the buffered aqueous solution is substantially free of primary amines. In another embodiment, the reaction occurs in an aqueous buffered solution in which the oligomeric nucleotide or mimic thereof is soluble. Soluble means more than ½ mg oligomeric nucleotide or mimic thereof per mL of aqueous buffered solution (i.e. ½ mg/mL aqueous buffered solution).

In another embodiment, the oligomeric nucleotide or mimic thereof is provided in excess of the crosslinking reagent-targeting species complex. The method further includes removing any excess unreacted oligomeric nucleotide or mimic thereof. Any excess unreacted oligomeric nucleotide or mimic thereof may be removed by any conventional means known to one of ordinary skill in the art

It should be appreciated that an aspect of the invention provides reacting the functional group of the crosslinking reagent with the targeting species and reacting the another functional group of the crosslinking reagent with the oligomeric nucleotide or mimic thereof simultaneously or sequentially. In a particular embodiment, reacting the functional group with the targeting specie occurs before reacting the another functional group with the oligomeric nucleotide or mimic thereof.

The crosslinking reagent includes at least one crosslinking reagent selected from a group consisting of a one carbon or long polymeric species; a C₁-C₂₅₀ length carbon with at least one heteroatom selected from O, S, N, P, and optionally substituted with halogen atoms; an oligomeric or polymeric species made of natural or synthetic monomers; and an oligomeric or polymeric moiety selected from a pharmacologically acceptable oligomer or polymer composition, an oligo- or poly-amino acid, peptide, saccharide, a nucleotide, and an organic moiety with 1-250 carbon atoms, either individually or in combination thereof, wherein the organic moiety with 1-250 carbon atoms optionally comprises at least one heteroatom.

In a particular embodiment, the functional group and the another functional group includes at least one member selected from a group consisting of activated esters (such as N-hydroxysuccinimide ester, pentafluorophenyl ester), a phosphoramidite, an isocyanate, an isothiocyanate, an aldehyde, an acid chloride, a sulfonyl chloride, a maleimide, an alkyl halide, an amine, a phosphine, a phosphate, an alcohol, a thiol, and combinations thereof. In a more particular embodiment, the functional group comprises N-hydroxysuccinimide ester and the another functional group comprises maleimide.

Described below is a general example of how targeting species 220 are conjugated or otherwise attached to the second oligomeric nucleotide or mimic 210 thereof to produce the pretargeting conjugate 200. For example, the conjugation or attachment can be effected via an amide bond or a disulfide bond. Targeting species that are polypeptides, such as an antibody or an antibody fragment, are conjugated to the second oligomeric nucleotide or mimic thereof via amide bonds between free amino groups of lysine residues present in the antibody or the antibody fragment and the terminal carboxylic group of the second oligomeric nucleotide or mimic thereof. Alternatively, amide bonds can be formed between free carboxylic groups of glutamic acid or aspartic acid residues present in the antibody or the antibody fragment and the terminal amino group of the second oligomeric nucleotide or mimic thereof. The conjugation process can be carried out by contacting the antibody or antibody fragment and the second oligomeric nucleotide or mimic thereof at pH in the range from about 7 to about 9.5 in a buffer, at or near room temperature. At the end of the reaction period, the conjugate is separated from the unreacted low molecular-weight materials by, for example, size exclusion chromatography and/or dialysis.

The second oligomeric nucleotide or mimic thereof can also be conjugated to the antibody or antibody fragment via disulfide bonds formed with cysteine residues present in the antibody or antibody fragment. In this case, a cysteine residue is first attached to an end of the second oligomeric nucleotide or mimic thereof to provide a mercapto group thereto. The second oligomeric nucleotide or mimic thereof, as modified, is then reacted with the antibody or antibody fragment to produce the pretargeting conjugate as above.

Alternatively, carbohydrate moieties present in the antibody or antibody fragment may be oxidized mildly, such as with sodium metaperiodate at or near room temperature. Unreacted sodium metaperiodate may be decomposed with ethylene glycol. The oxidized antibody or antibody fragment is then separated from low molecular-weight materials, for example, by size exclusion chromatography, and subsequently reacted with the second oligomeric nucleotide or mimic thereof to produce the pretargeting conjugate.

Methods for Diagnosing or Treating Diseases using Pretargeting Strategy

With reference to FIG. 3, next will be described a method for diagnosing a disease condition by preferentially delivering a diagnostic active agent to the site of the disease. FIG. 3 is a flow chart of the method. The method includes, at Step 305 administering a pretargeting conjugate to a subject. At Step 315, the pretargeting conjugate is allowed to localize at the target. Step 325 includes administering an active agent-labeled species to the subject, wherein the active agent-labeled species comprises a diagnostic active agent conjugated to the first oligomeric nucleotide or mimic thereof sequence.

In one embodiment, the targeting species is an antibody or a fragment thereof that binds to an antigen present at the target, which can be a diseased cell or tissue, or a marker substance produced by the diseased cell or tissue. The diagnostic active agent is a moiety that generate a unique signal that is recognizable by diagnostic medical imaging techniques, such as MRI, PET, SPECT, X-ray imaging, CT, ultrasound imaging, or optical imaging.

In another embodiment, the diagnostic active agent is any of the diagnostic active agents described herein above, either individually or in combination thereof. In a particular embodiment, the diagnostic active agent is a radioisotope, drug, toxin, fluorescent dye activated by nonionizing radiation, hormone, hormone antagonist, receptor antagonist, enzyme or proenzyme activated by another agent, autocrine, or cytokine.

In one aspect, the active agent-labeled species comprises a poly(amino acid) (e.g., as polylysine), wherein at least 90 percent of the lysine residues are conjugated to chelating moieties, (e.g., DTPA), which form coordination complex with paramagnetic ions, (e.g., Gd³⁺) for MRI application. The active agent-labeled species may be administered into the patient at a dose from about 0.01 to about 0.05 moles Gd/kg of body weight of the patient. An MRI system that can be used for practicing a method of the invention is disclosed in U.S. Pat. No. 6,235,264. The pair of pharmaceuticals of the invention is formulated with a physiologically acceptable carrier, such as an intravenous fluid, for intravenously administering into the patient. These pharmaceuticals may also be administered orally under appropriate circumstances.

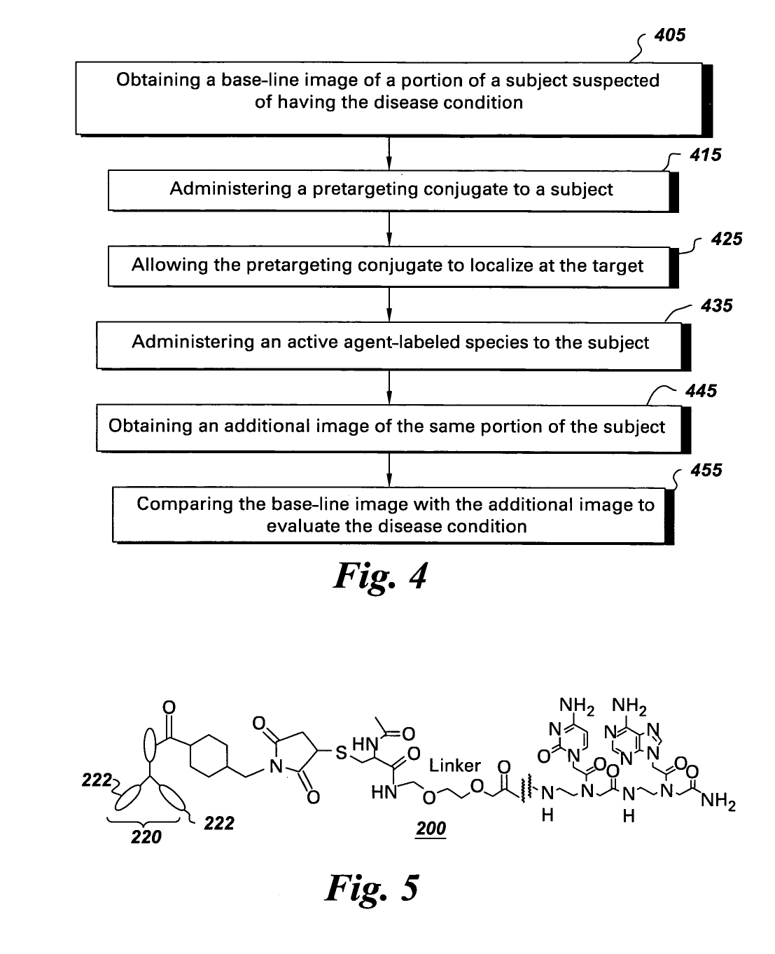

With reference to FIG. 4, next will be described another method for diagnosing a disease condition. The method include, at Step 405, obtaining one or more base-line image of a portion of a subject suspected of having the disease condition. Image as used herein includes signals as well as any other visual representation of the spatial distribution (or location) of an object. In one embodiment, an image consists of an array (of more than one dimension), where the values of the array typically represent an intensity associated with a spatial coordinate in two or three dimensions.

Step 415 includes administering a pretargeting conjugate to the subject. Step 425 includes allowing the pretargeting conjugate to localize at the target. Step 435 includes administering an active agent-labeled species to the subject. Step 445 includes obtaining one or more additional image of the same portion of the subject. Step 455 includes comparing the base-line image with the additional image to evaluate the disease condition. The step of obtaining additional images to evaluate the disease condition may be repeated at different time intervals as desired. Thus, it should be appreciated that one or more base line images may be compared with one or more additional images or the additional images may be compared with each other.

Another aspect of the invention provides a method for assessing an effectiveness of a prescribed regimen for treating a disease condition that is characterized by an overproduction or underproduction of a disease-specific substance or biomarker. The method includes (i) obtaining a base-line image of a portion of a subject suspected of having the disease condition; (ii) administering a pretargeting conjugate to the subject; (iii) allowing the pretargeting conjugate to localize at the target; (iv) administering an active agent-labeled species to the subject; (v) obtaining a pre-treatment image coming from the same portion of the subject; (vi) treating the disease condition in the subject with a prescribed regimen; (vii) repeating steps (ii), (iii), and (iv); and (viii) obtaining a post-treatment image coming from the same portion of the subject as in step (v).

The method may further comprise comparing the post-treatment image to the pre-treatment image to assess the effectiveness of the prescribed regimen, wherein a change in image contrast during a course of the prescribed regimen indicates that the treatment has provided benefit. The method may also further comprise comparing the post-treatment image to the baseline image to assess the effectiveness of the prescribed regimen, wherein a change in image contrast or signals during a course of the prescribed regimen indicates that the treatment has provided benefit. The method may also further comprising repeating steps (vii) and (viii) at predetermined time intervals during the course of treating the disease condition.

In various aspects of the methods, any one of the pretargeting conjugates and active agent-labeled species that are described above can be chosen to suit the particular circumstances and disease.

During the course of the treatment of the disease, a decreased signal obtained from the imaging technique (compared to a base-line signal obtained before the treatment) of, for example, 10 percent or more can signify that the treatment has conferred some benefit. In another embodiment, a decreased signal obtained from the imaging technique (compared to a base-line signal obtained before the treatment) of, for example, 20 percent or more can signify that the treatment has conferred some benefit. The prescribed regimen for treating the disease can be, for example, treatment with drugs, radiation, or surgery.

In one aspect, the invention provides a kit that comprises the active agent-labeled species and the pretargeting conjugate kept separately before use for purposes of diagnosing or treating diseases.

The compounds, for example, the active agent-labeled species, the pretargeting conjugate, or both, can be incorporated into pharmaceutical compositions suitable for administration into a subject, which pharmaceutical compositions comprise a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible with the subject. Particularly, the carrier is suitable for intravenous, intramuscular, subcutaneous, or parenteral administration (e.g., by injection). Depending on the route of administration, the active agent-labeled species, the pretargeting conjugate, or both, may be coated in a material to protect the compound or compounds from the action of acids and other natural conditions that may inactivate the compound or compounds.

In yet another embodiment of the invention, the pharmaceutical composition comprising the active agent-labeled species, the pretargeting conjugate, or both, and a pharmaceutically acceptable carrier can be administered by combination therapy, i.e., combined with other agents. For example, the combination therapy can include a composition of the invention with at least a therapeutic agent or drug, such as an anti-cancer or an antibiotic. Exemplary anti-cancer agents include cis-platin, adriamycin, and taxol. Exemplary antibiotics include isoniazid, rifamycin, and tetracycline.

### EXAMPLES

Pretargeting Conjugate of Ab-Oligomer Bioconjugation

FIG. 5 is a schematic representation of a pretargeting conjugate comprising an antibody-peptide nucleic acid (Ab-PNA) as described below. The targeting species, an anti-carcinoembryonic antigen (CEA) mouse monoclonal antibody T84.66 (0.8 mg/ml) was incubated with succinimidyl 4-[N-maleimido-methyl]cyclohexane-1-carboxylate (SMCC) cross-linking reagent (Pierce, Rockford, Ill.) in PBS buffer (pH 7.5) at a molar antibody to SMCC ratio of 1:50. After 90 min. at room temperature, the amine coupling reaction was quenched with 10 mM Tris-HCl (pH 7.5) for 15 min. Excess cross-linking reagent was removed by sequential spin filtration of the reaction mixture with 0.1 M sodium phosphate, 0.1 M NaCl, 5 mM EDTA, pH 6.5 buffer using an Amicon Ultra 30K MWCO spin filter (3×, 14,000×g). The antibody-containing fraction was collected and the maleimide content was measured by back titration with cysteine and Eliman's reagent (Pierce) as previously described. Antibody concentration was determined using the Bicinchoninic acid (BCA) protein assay (Pierce) and bovine gamma globulin (Pierce) as the calibration standard. The ratio of maleimide/Ab was further validated by matrix assisted laser desorption ionization time of flight mass spectrometry (MALDI-TOF MS) of the intact IgG.

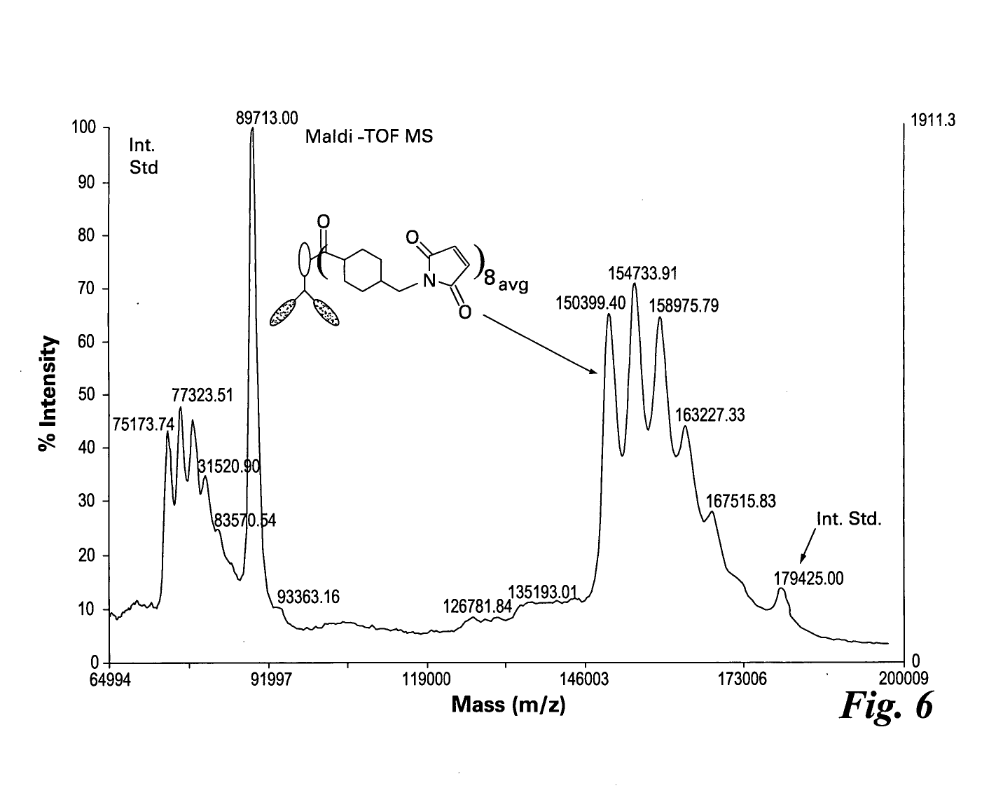

The thiol-derivatized PNA intended for conjugation was incubated with immobilized tris[2-carboxyethyl]phosphine (TCEP) agarose gel following the manufacturer's protocol (Pierce). Immediately following reduction, the free thiol-containing PNA was mixed with the maleimide derivatized mAb (0.4-0.8 mg/ml) at a molar Ab to oligomer ratio of 1:10 and incubated overnight at 4° C. Excess PNA was subsequently removed by sequential spin filtration of the reaction mixture with 0.1 M sodium phosphate, pH 7.5 buffer using an Amicon Ultra 30K MWCO spin filter (3×, 14,000×g). The pretargeting conjugate (FIG. 5) was characterized by Maldi-TOF MS and UV-Vis spectroscopy to determine the average number of oligomers per antibody and subsequently stored at 4° C. for further use. FIG. 6 is a Maldi-time of flight (TOF) MS spectrum of the Ab-PNA pretargeting conjugate showing the average number of oligomers per antibody (n=1-5) for a PNA oligomer with a given molecular weight (MW=4423 Da.). Int. Std. means internal standard.

In-Vitro Affinity Characterization

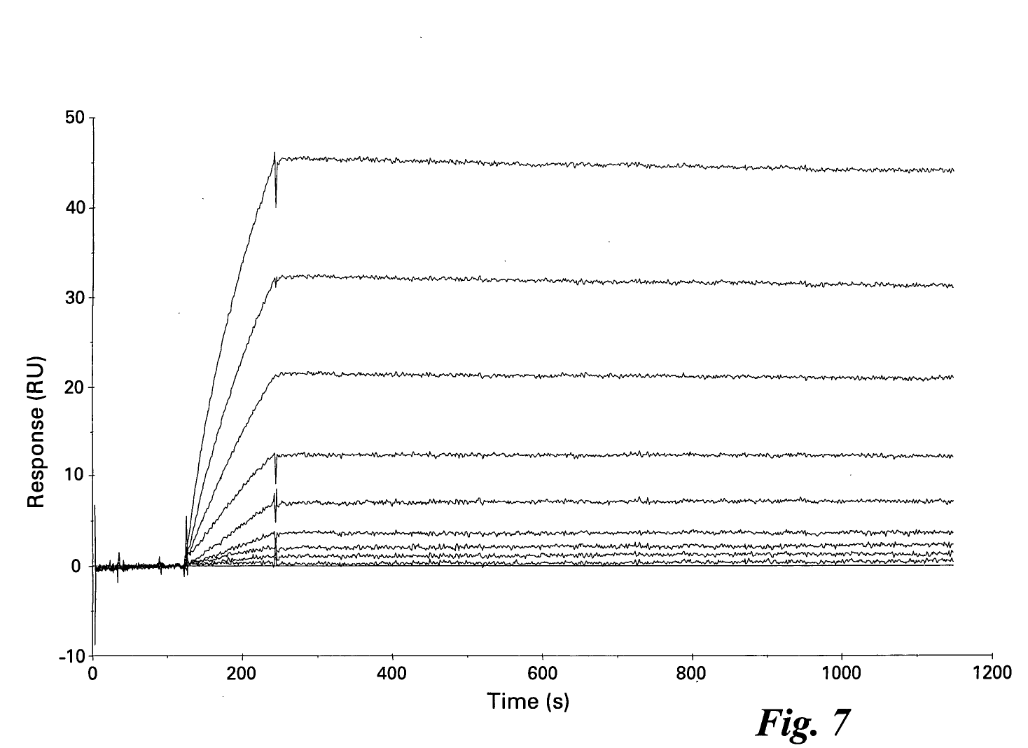

Binding interactions between 1) the Ab-PNA conjugate (i.e. pretargeting conjugate) and the biomarker, and 2) the Ab-PNA conjugate (i.e. pretargeting conjugate) and the TETA-cPNA molecule (i.e. species to be labeled with the active agent) were conducted using surface plasmon resonance detection (Biacore™). Biacore™ is a surface plasmon resonance instrument for conducting kinetic binding studies. The pretargeting conjugate was covalently attached to a CM-5 (Biacore™) dextran-functionalized sensor chip pre-equilibrated with HBS-EP buffer (10 ul/min) and activated with 1-ethyl-3-[3-dimethylaminopropyl]carbodiimide hydrochloride (EDC) and N-hydroxysuccinimide (NHS). The pretargeting conjugate in 10 mM sodium acetate (pH 5) was injected onto the activated sensor chip until the desired immobilization level was achieved (˜50 RU). Residual activated groups on the sensor chip were blocked by injection of ethanolamine (1 M, pH 8.5). Non-covalently bound pretargeting conjugate were removed by repeated (5×) washing with 50 mM NaOH. Prior to the kinetic study, binding of the biomarker, carcinoembryonic antigen (CEA), was tested and a surface stability experiment was performed to ensure adequate removal of the bound biomarker and regeneration of the sensor chip following treatment with 50 mM NaOH. Kinetic measurements were conducted over a range of 8 analyte concentrations (0-200 nM) with a 5-15 min association period, and a 15-45 min. dissociation period. FIG. 7 is a Biacore™ kinetic study demonstrating the binding of the Ab-PNA pretargeting conjugate to a target (CEA). Raw data was analyzed using the BiaEval Biacore™ software which handles numerical curve fitting of a response curve to generate the reaction rate constants. The raw data was fitted to 1:1 Langmuir binding model, yielding a K_{D}˜10⁻¹⁰ M for the pretargeting conjugate-biomarker interaction, demonstrating a high binding affinity of the antibody for the biomarker despite the chemical conjugation to the antibody as shown in FIG. 7 and described in Table 1 below.

TABLE 1

| PNA-Ab | |
|---|---|
| kₒₙ = 60.5 (1/mMs) | K_{A} = 1.4e9 (1/M) |
| K_{off} = 4.4e−5 (1/s) | K_{D} = 7.3e−10 (M) |

T84.66 K_{D} ˜3.8 × 10⁻¹¹ M (Shively, 1990)

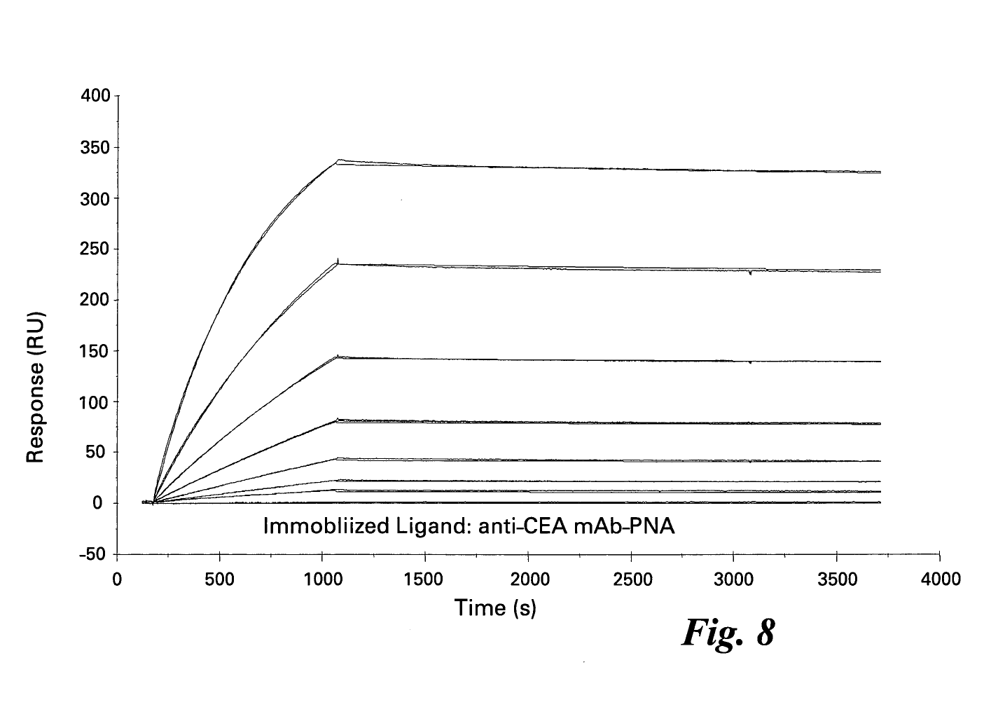

To test cPNA hybridization to the Ab-PNA pretargeting conjugate (i.e. binding of the active-labeled species to the pretargeting agent), binding & kinetic studies similar to the experiments described above were also conducted. A specific binding interaction was observed between the Ab-PNA (i.e. pretargeting conjugate) and the cPNA oligomer (i.e. species to be labeled with active agent) that was not observed with a non-complementary PNA oligomer of the same length. FIG. 8 is a Biacore™ kinetic study demonstrating the coupling of an active agent labeled species with the ab-PNA pretargeting conjugate. Kinetic measurements on the immobilized pretargeting conjugated (Ab-PNA) with free TETA labeled cPNA, yielded a K_{D}˜10⁻⁹ M, indicating a high binding affinity between the TETA-cPNA and the PNA-Ab as shown in FIG. 8 and described in Table 2 below. These results were consistent with literature reported K_{D} values for unmodified PNA-cPNA using similar length oligomers.

TABLE 2

| | |
|---|---|
| kₒₙ = 3.3 (1/mMs) | K_{A} = 3.43e8 (1/M) |
| K_{off} = 9.62e−6 (1/s) | K_{D} = 2.92e−9 (M) |

T84.66 K_{D} ˜3.8 × 10⁻¹¹ M (Shively, 1990)

Active Agent Labeled Species

Described below is the Cu-oligomer bioconjugation of the following active agent-linker:

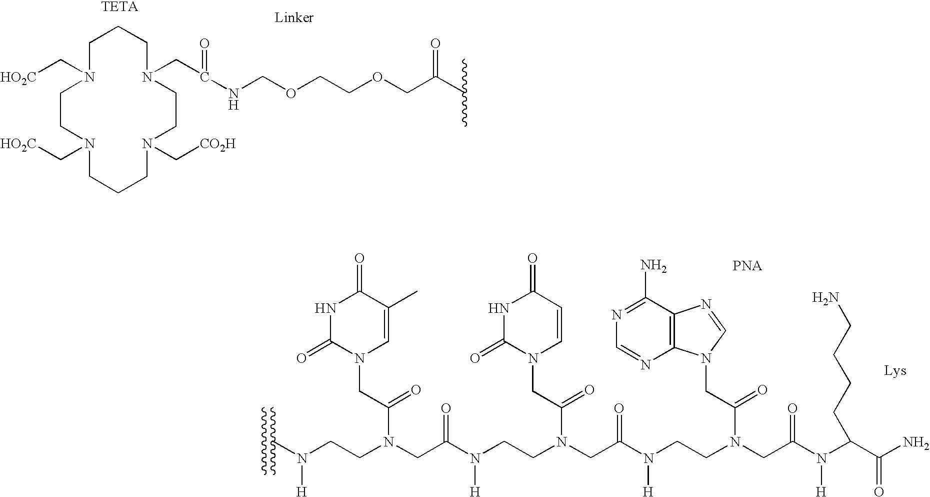

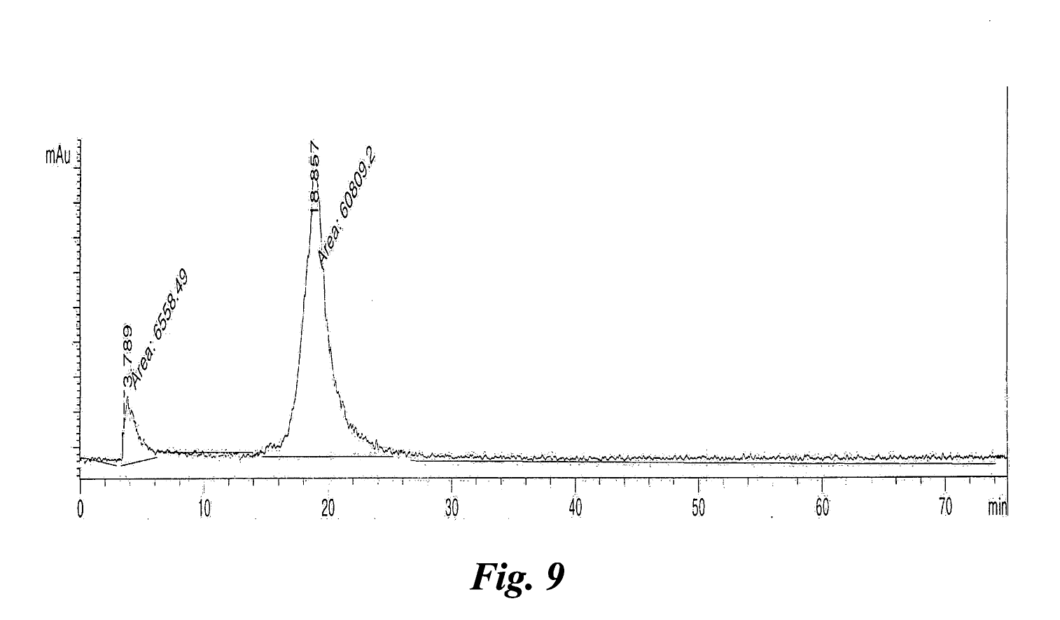

To a test tube containing 25 μg of TETA-cPNA in 25 μL 0.1 M ammonium acetate buffer, pH 5.5, was added 25 μCi ⁶⁴Cu(OAc)₂ in 25 μL 0.1 M H₄OAc buffer, pH 5.5. The tube was heated in a lead-shielded water bath for 1 h at 50° C. The reaction was cooled to room temperature, at which time 5.6 μL 10 mM EDTA was added, giving a solution 1 mM in EDTA. This solution was left to stand at room temperature for 15 minutes, at which time a 1 μCi aliquot was chromatographed on RP-18 HPLC. Solvent A was 0.1% aqueous trifluoroacetic acid (TFA) and Solvent B was 0.1% TFA in MeCN. The gradient was as follows (t(min)/% B): 0/5, 15/15, 50/60, 51/100, 65/100, 66/5, 75/5. FIG. 9 is a radio-high performance liquid chromatogram (HPLC) of a Cu-64 labeled 1,4,8,11-tetraazacyclotetradecane-N,N′,N″,N′″-tetraacetic acid-cPNA active agent labeled species. The active agent labeled species eluted with a retention time of 18.9 minutes in a 90% radiochemical yield (FIG. 9).

In another example, a sample of purified oligonucleotide mimic conjugated to an appropriate chelator (TETA, for example) is labeled with an appropriate radioisotope (e.g. ⁶⁴Cu) in the following manner. A small amount (10-100 μg) is dissolved, preferably in water or pH 5.5 acetate buffer, and incubated at RT or elevated temperature for 1 hour with an appropriate amount of radioactivity (from about 10 μCi to about 4 mCi). The reaction is quenched by addition of a volume 10 mM EDTA to give a solution of a 1 mM EDTA final concentration. The product is evaluated for its radiochemical purity by iTLC and/or HPLC and is purified, if necessary, by size exclusion chromatography or HPLC, to yield the pure radiolabeled oligonucleotide mimic. An example with TETA and ⁶⁴Cu is shown below with the macrocyclic chelator, TETA, conjugated to the biomolecule of interest via the N-terminus or, alternatively, at the ε-amine of a lysine side chain.

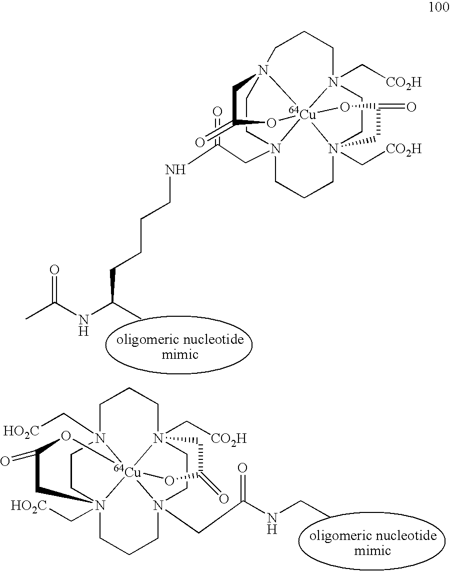

In this manner, the oligomeric nucleotide mimic may be a labeled with diagnostic radionuclides such as indium-111, gallium-67, gallium-68, ruthenium-97, technetium-99m, cobalt-57, cobalt-58, chromium-51, iron-59, thallium-201, ytterbium-169, and copper-64.

Alternatively, the oligomeric nucleotide mimic may be labeled with radioactive non-metals such as radioisotopes of iodine (such as I-123, I-124, I-125) or F-18 using a prosthetic group approach. Through the N-terminus or through the E-amine of a lysine side chain, iodobenzoic acid or fluorobenzoic acid may be chemically linked to the mimic. A sample of preformed iodo- or fluorobenzoic acid activated ester is incubated with a small amount (10-100 μg) of oligonucleotide mimic in an appropriate solvent (likely organic), with a final activity ranging from about 10 μCi to about 4 mCi. The radiolabeled species is purified using HPLC to give the final product as a radiochemically pure species. A particular embodiment, with ¹⁸F as the diagnostic active agent, is shown below.

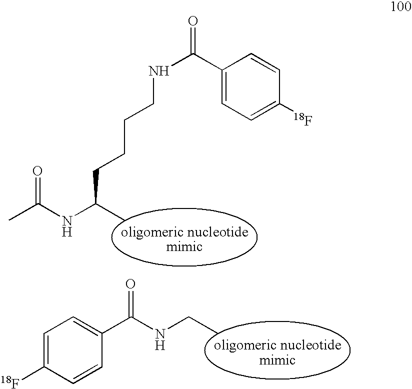

While the invention has been described in detail in connection with only a limited number of aspects, it should be readily understood that the invention is not limited to such disclosed aspects. Rather, the invention can be modified to incorporate any number of variations, alterations, substitutions or equivalent arrangements not heretofore described, but which are commensurate with the spirit and scope of the invention. Additionally, while various embodiments of the invention have been described, it is to be understood that aspects of the invention may include only some of the described embodiments. Accordingly, the invention is not to be seen as limited by the foregoing description, but is only limited by the scope of the appended claims.

What is claimed is:

1. A method of making a pretargeting conjugate comprising:
(i) reacting a functional group of a crosslinking reagent with a targeting species to form a crosslinking reagent-targeting species complex;
(ii) reacting another functional group of the crosslinking reagent with an oligomeric nucleotide or mimic thereof to form a pretargeting conjugate.

2. The method of claim 1, wherein reacting the functional group of the crosslinking reagent with the targeting species comprises incubating the functional group of the crosslinking reagent with the targeting species.

3. The method of claim 1, wherein reacting the functional group of the crosslinking reagent with the targeting species comprises a covalent bond between the crosslinking reagent and the targeting species.

4. The method of claim 1, further comprising providing the cross linking reagent in excess of the targeting species.

5. The method of claim 4, further comprising removing any excess unreacted crosslinking reagent.

6. The method of claim 1, wherein reacting the functional group of the crosslinking reagent with the targeting species occurs in a buffered aqueous solution maintained at a pH from about 6.5 to about 7.5.

7. The method of claim 6, wherein the pH is about neutral.

8. The method of claim 6, wherein the buffered aqueous solution is substantially free of primary amines.

9. The method of claim 1, further comprising inhibiting the reaction between the functional group of the crosslinking reagent and the targeting species.

10. The method of claim 9, wherein inhibiting the reaction between the functional group of the crosslinking reagent and the targeting species comprises providing a primary amine.

11. The method of claim 1, wherein reacting the another functional group of the crosslinking reagent with the oligomeric nucleotide or mimic thereof comprises incubating the crosslinking reagent with the oligomeric nucleotide or mimic thereof.

12. The method of claim 1, wherein reacting the another functional group of the crosslinking reagent with the oligomeric nucleotide or mimic thereof comprises a covalent bond between the crosslinking reagent and the oligomeric nucleotide or mimic thereof.

13. The method of claim 1, wherein reacting the another functional group of the crosslinking reagent with the oligomeric nucleotide or mimic thereof occurs in a buffered aqueous solution maintained at a pH from about 6.5 to about 7.5.

14. The method of claim 1, wherein reacting the another functional group of the crosslinking reagent with the oligomeric nucleotide or mimic thereof occurs in an aqueous solution in which the oligomeric nucleotide or mimic thereof is soluble.

15. The method of claim 1, further comprising providing the oligomeric nucleotide or mimic thereof in excess of the crosslinking reagent-targeting species complex.

16. The method of claim 15, further comprising removing any excess unreacted oligomeric nucleotide or mimic thereof.

17. The method of claim 1, wherein the crosslinking reagent comprises at least crosslinking reagent selected from a group consisting of:
at least a carbon long polymeric species;
a C₁-C₂₅₀ length carbon with at least one heteroatom selected from a group consisting of O, S, N, and P, and optionally substituted with a halogen;
an oligomeric or polymeric species made of natural or synthetic monomers; and
an oligomeric or polymeric moiety selected from a group consisting of a pharmacologically acceptable oligomer or polymer composition, an oligo- or poly-amino acid, peptide, a saccharide, a nucleotide, an organic moiety with 1-250 carbon atoms, and combination thereof;
wherein the organic moiety with 1-250 carbon atoms optionally comprises at least one heteroatom.

18. The method of claim 1, wherein the functional group and the another functional group comprises at least one member selected from a group consisting of activated esters, a phosphoramidite, an isocyanate, an isothiocyanate, an aldehyde, an acid chloride, a sulfonyl chloride, a maleimide, an alkyl halide, an amine, a phosphine, a phosphate, an alcohol, a thiol, and combinations thereof.

19. The method of claim 1, wherein the functional group comprises N-hydroxysuccinimide ester.

20. The method of claim 1, wherein the another functional group comprises maleimide.

21. The method of claim 1, wherein reacting the functional group of the crosslinking reagent with the targeting species and reacting the another functional group of the crosslinking reagent with the oligomeric nucleotide or mimic thereof occurs simultaneously.

22. The method of claim 1, wherein reacting the functional group of the crosslinking reagent with the targeting species and reacting the another functional group of the crosslinking reagent with the oligomeric nucleotide or mimic thereof occurs sequentially.

23. The method of claim 22, wherein reacting the functional group of the crosslinking reagent with the targeting species occurs before reacting the another functional group of the crosslinking reagent with the oligomeric nucleotide or mimic thereof.

24. The method of claim 1, wherein the oligomeric nucleotide or mimic thereof is a sequence having a formula of: 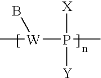
wherein X and Y independently at each occurrence is selected from a group consisting of O, S, OR, BH₃, and NR¹R²;
wherein R independently at each occurrence is selected from a group consisting of a C₁-C₆ alkyl;
wherein R¹ and R² independently at each occurrence is selected from a group consisting of H and C₁-C₆ alkyl;
wherein W independently at each occurrence is selected from a group consisting of an acyclic moiety, a carbocyclic moiety, and a natural or a synthetic sugar moiety;
wherein B independently at each occurrence comprises a natural or synthetic nitrogen-comprising heterocyclic base; and
wherein n is an integer in a range from about 4 to about 100.

25. A method of making a pretargeting conjugate comprising:
(i) reacting a functional group of a crosslinking reagent with a targeting species to form a crosslinking reagent-targeting species complex in a buffered aqueous solution at about a neutral pH substantially free of primary amines;
(ii) reacting another functional group of the crosslinking reagent with an oligomeric nucleotide or mimic thereof to form the pretargeting conjugate.
